(19) United States
(12) Patent Application Publication
(10) Pub. No.: US 2017/0042105 A1
(43) Pub. Date: Feb. 16, 2017

(54) INTEGRATED BAMBOO PLANTATIONS WITH NON-BAMBOO VEGETATION AND BAMBOO

(71) Applicant: EcoPlanet Bamboo Group, Barrington, IL (US)

(72) Inventors: Camille REBELO, Nairobi (KE); Troy WISEMAN, Barrington Hills, IL (US)

(21) Appl. No.: 14/825,772

(22) Filed: Aug. 13, 2015

Related U.S. Application Data
(60) Provisional application No. 62/203,068, filed on Aug. 10, 2015.

(51) Int. Cl. A01H 5/04; A01G 1/00; B01J 20/20; D21H 11/12; D04H 3/013

(52) U.S. Cl. CPC A01H 5/04; D21H 11/12; D04H 3/013; B01J 20/20; A01G 1/001

(57) ABSTRACT

The invention is directed to a bamboo plantation where the bamboo is integrated with pre-existing non-bamboo vegetation. In some aspects, the invention relates to a high-yielding bamboo plantation. In some aspects, the invention relates to a bamboo plantation grown on degraded or marginal land. In some aspects, the invention relates to the conversion of degraded or marginal land to a high-yielding bamboo plantation.

This application claims the benefit of U.S. Provisional Application No. 62/203,068, filed Aug. 10, 2015, the contents of which is hereby incorporated by reference in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

Bamboo fiber has the ability to transform the timber industry and reverse the current trend of global deforestation. Maximizing bamboo plant yield is critical for the industrialization of bamboo as a commercial crop. Commercial growth of bamboo has traditionally focused on clearing of native ecosystems to grow a single species of bamboo under a monoculture system. The clearance of trees, forests, or other native vegetation for monoculture is detrimental to the environment.

There is a need for high-yield bamboo plantations that can be grown sustainably.

### SUMMARY OF THE INVENTION

The invention relates to a bamboo plantation where the bamboo is integrated with pre-existing non-bamboo vegetation. In some aspects, the invention relates to a high-yielding bamboo plantation. In some aspects, the invention relates to a bamboo plantation grown on degraded or marginal land. In some aspects, the invention relates to the conversion of degraded or marginal land to a high-yielding bamboo plantation.

In certain aspects, the invention relates to a bamboo plantation comprising pre-existing non-bamboo vegetation and rows of sympodial bamboo plants, wherein the pre-existing vegetation comprises at least 10% of the total area of the plantation, and wherein the sympodial bamboo plants are planted at least 1 meter apart, and wherein the rows of sympodial bamboo plants are spaced at least 2 meters apart. In some embodiments, the sympodial bamboo plants are planted at an average density over the total area of the plantation of at least 75 plants per acre. In some embodiments, the sympodial bamboo plants comprise one species of sympodial bamboo. In some embodiments, the total area of the plantation is at least 1 acre. In some embodiments, the sympodial bamboo plants comprise more than one species of sympodial bamboo, wherein one species of sympodial bamboo is planted in an area of the plantation and each area is at least 1 acre. In some embodiments, the pre-existing non-bamboo vegetation comprises at least 20% of the total area of the plantation. In some embodiments, the pre-existing non-bamboo vegetation comprises at least 30% of the total area of the plantation. In some embodiments, the pre-existing non-bamboo vegetation comprises at least 40% of the total area of the plantation. In some embodiments, the pre-existing non-bamboo vegetation comprises at least 50% of the total area of the plantation. In some embodiments, the density of pre-existing non-bamboo vegetation is not uniform within the plantation. In some embodiments, the pre-existing non-bamboo vegetation is not removed to plant the sympodial bamboo plant. In some embodiments, at least one area of the plantation comprises only pre-existing non-bamboo vegetation. In some embodiments, the sympodial bamboo plants are planted from 1 to 10 meters apart. In some embodiments, the rows of sympodial bamboo plants are spaced from 2 to 8 meters apart. In some embodiments, the plantation yields at least 60 metric tons of raw green bamboo biomass per hectare per year.

In some embodiments, the plantation yields at least 20% more raw green bamboo biomass per hectare per year than a bamboo plantation comprising only bamboo plants. In some embodiments, the plantation yields at least 20% more raw green bamboo biomass per hectare per year than a bamboo plantation comprising less than 10% non-bamboo vegetation. In some embodiments, the plantation yields at least 30% more raw green bamboo biomass per hectare per year. In some embodiments, the plantation yields at least 40% more raw green bamboo biomass per hectare per year. In some embodiments, the plantation yields at least 50% more raw green bamboo biomass per hectare per year. In some embodiments, the plantation yields at least 60% more raw green bamboo biomass per hectare per year. In some embodiments, the plantation yields 20% to 60% more raw green bamboo biomass per hectare per year. In some embodiments, the plantation yields 20% to 30% more raw green bamboo biomass per hectare per year. In some embodiments, the plantation yields 30% to 40% more raw green bamboo biomass per hectare per year. In some embodiments, the plantation yields 40% to 50% more raw green bamboo biomass per hectare per year. In some embodiments, the plantation yields 50% to 60% more raw green bamboo biomass per hectare per year.

In some embodiments, the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

In certain aspects, the invention relates to a method of planting a bamboo plantation, comprising planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area of the plantation. In some embodiments, the sympodial bamboo plants are planted at a density of at least 75 plants per acre. In some embodiments, one species of sympodial bamboo is planted. In some embodiments, more than one species of sympodial bamboo is planted, and wherein one species of sympodial bamboo is planted in an area of the plantation and each area is at least 1 acre. In some embodiments, the pre-existing non-bamboo vegetation comprises at least 20% of the total area of the plantation. In some embodiments, the pre-existing non-bamboo vegetation comprises at least 30% of the total area of the plantation. In some embodiments, the pre-existing non-bamboo vegetation comprises at least 40% of the total area of the plantation. In some embodiments, the pre-existing non-bamboo vegetation comprises at least 50% of the total area of the plantation. In some embodiments, the density of pre-existing non-bamboo vegetation is not uniform within the plantation. In some embodiments, the pre-existing non-bamboo vegetation is not removed to plant the sympodial bamboo plant. In some embodiments, the sympodial bamboo plants are planted from 1 to 10 meters apart. In some embodiments, the rows of sympodial bamboo plants are spaced from 2 to 8 meters apart.

In some embodiments, the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

In certain aspects, the invention relates to a method of growing sympodial bamboo, comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In some embodiments, the bamboo yields at least 60 tons of raw green bamboo biomass per hectare per year.

In some embodiments, the bamboo yields at least 20% more raw green bamboo biomass per hectare per year than a bamboo plantation comprising only bamboo plants. In some embodiments, the bamboo yields at least 20% more raw green bamboo biomass per hectare per year than a bamboo plantation comprising less than 10% non-bamboo vegetation. In some embodiments, the bamboo yields at least 30% more raw green bamboo biomass per hectare per year. In some embodiments, the bamboo yields at least 40% more raw green bamboo biomass per hectare per year. In some embodiments, the bamboo yields at least 50% more raw green bamboo biomass per hectare per year. In some embodiments, the bamboo yields at least 60% more raw green bamboo biomass per hectare per year. In some embodiments, the bamboo yields 20% to 60% more raw green bamboo biomass per hectare per year. In some embodiments, the bamboo yields 20% to 30% more raw green bamboo biomass per hectare per year. In some embodiments, the bamboo yields 30% to 40% more raw green bamboo biomass per hectare per year. In some embodiments, the bamboo yields 40% to 50% more raw green bamboo biomass per hectare per year. In some embodiments, the bamboo yields 50% to 60% more raw green bamboo biomass per hectare per year.

In some embodiments, the sympodial bamboo plants are planted at a density of at least 75 plants per acre. In some embodiments, one species of sympodial bamboo is planted. In some embodiments, more than one species of sympodial bamboo is planted, and wherein one species of sympodial bamboo is planted in an area and each area is at least 1 acre. In some embodiments, the pre-existing non-bamboo vegetation comprises at least 20% of the total area of the plantation. In some embodiments, the pre-existing non-bamboo vegetation comprises at least 30% of the total area of the plantation. In some embodiments, the density of pre-existing non-bamboo vegetation is not uniform. In some embodiments, the pre-existing non-bamboo vegetation is not removed to plant the sympodial bamboo plant. In some embodiments, the sympodial bamboo plants are planted from 1 to 10 meters apart. In some embodiments, the rows of sympodial bamboo plants are spaced from 2 to 8 meters apart.

In some embodiments, the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica. In some embodiments, the sympodial bamboo is planted on degraded land. In some embodiments, growing sympodial bamboo on the degraded land restores the ecosystem functions of the degraded land.

In certain aspects, the invention relates to bamboo pulp prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to paper prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to textiles prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to activated carbon prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to engineered timber prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants. In some embodiments, the engineered timber is a composite timber, a plywood or a veneer.

### BRIEF DESCRIPTION OF THE DRAWINGS

The patent or application file contains at least one drawing or photograph executed in color. Copies of this patent or patent application publication with color drawing(s) will be provided by the United States Patent and Trademark Office upon request and payment of the necessary fee.

FIG. 1 shows a schematic of an embodiment of a bamboo plantation where the bamboo is integrated with pre-existing non-bamboo vegetation. FIG. 1 shows rows of sympodial bamboo plants integrated with the pre-existing non-bamboo vegetation. In FIG. 1, a conserved forest is shown as an example of pre-existing non-bamboo vegetation.

FIG. 2 shows a schematic of an embodiment of a bamboo plantation where the bamboo is integrated with pre-existing non-bamboo vegetation. FIG. 2 shows rows of sympodial bamboo plants integrated with the pre-existing non-bamboo vegetation. In FIG. 2, standing trees are shown as an example of pre-existing non-bamboo vegetation.

FIG. 3 shows a schematic of an embodiment of a bamboo plantation where the bamboo is integrated with pre-existing non-bamboo vegetation. FIG. 3 shows sympodial bamboo plants planted at least 1 meter apart, and wherein the rows of sympodial bamboo plants are spaced at least 2 meters apart.

FIG. 4 shows a schematic of an embodiment of a bamboo plantation where the bamboo is integrated with pre-existing non-bamboo vegetation. FIG. 4 shows sympodial bamboo plants planted at least 10 meters apart, and wherein the rows of sympodial bamboo plants are spaced at least 8 meters apart.

FIG. 5 shows an embodiment of a bamboo plantation in Nicaragua where the bamboo is integrated with pre-existing non-bamboo vegetation. FIG. 5 shows a 3 year old plantation where the sympodial bamboo Guadua aculeata plants have been planted 6.25 meters apart, and wherein the rows of sympodial bamboo plants are spaced at 6.25 meters apart.

FIG. 6 shows an embodiment of a bamboo plantation in Nicaragua where the bamboo is integrated with pre-existing non-bamboo vegetation. FIG. 6 shows a 2 year old plantation where the sympodial bamboo Guadua aculeata plants have been planted 5 meters apart, and wherein the rows of sympodial bamboo plants are spaced at 5 meters apart.

FIG. 7 shows an embodiment of a bamboo plantation where the bamboo is integrated with pre-existing non-bamboo vegetation. FIG. 7 shows a newly planted plantation where the sympodial bamboo Guadua aculeata plants have been planted at least 5 meters apart, and wherein the rows of sympodial bamboo plants are spaced at least 5 meters apart.

FIG. 8 shows an embodiment of a bamboo plantation where the bamboo is integrated with pre-existing non-bamboo vegetation. FIG. 8 shows an aerial view of a plantation where sympodial bamboo plants have been planted at a density of at least 175 plants per acre.

FIG. 9 shows an embodiment of a bamboo plantation in South Africa where the bamboo is integrated with pre-existing non-bamboo vegetation. FIG. 9 shows a 1 year old plantation where the sympodial bamboo Bambusa balcooa plants have been planted 5 meters apart, and wherein the rows of sympodial bamboo plants are spaced at 5 meters apart.

FIG. 10 shows an embodiment of a bamboo plantation in Ghana where the bamboo is integrated with pre-existing non-bamboo vegetation. FIG. 10 shows a newly planted plantation where the sympodial bamboo Dendrocalamus asper plants have been planted 5 meters apart, and wherein the rows of sympodial bamboo plants are spaced at least 3 meters meters apart.

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The invention relates to a bamboo plantation where the bamboo is integrated with pre-existing non-bamboo vegetation. In some aspects, the invention relates to a high-yielding bamboo plantation. In some aspects, the invention relates to a bamboo plantation grown on degraded or marginal land. In some aspects, the invention relates to the conversion of degraded or marginal land to a high-yielding bamboo plantation.

Bamboo as a Sustainable Fiber

Bamboo fiber has the ability to transform the timber industry and reverse the current trend of global deforestation. Bamboo fiber, woven, or in laminated form, can be used for plywood, composites and veneers. With a relatively long fiber length its pulp can also be used for a range of paper products, from sanitary paper, to copy paper to packaging. It can also be fed into the dissolving pulp industry with little change to machinery, and be used to replace cellulosic pulp with a yarn that exhibits a range of superior properties. Bamboo also has a calorific value equivalent to most woods, allowing it to provide renewable and distributed energy across all levels of processing, from simple charcoal to gasification.

Bamboo's advantages are multidimensional but focus around the grass's ability to produce high volumes of fiber per unit area, even when grown on degraded land. Furthermore, its fiber can be substituted in a diverse range of products, including, but not limited to paper, plywood, MDF board, textiles, and bioenergy.

Ecologically, bamboo has many unique attributes that enable it to have the potential to be one of the most sustainable sources of fiber or biomass on the planet. It can reverse land degradation, restore canopy cover and maintain soil productivity, water tables and carbon cycles. Described herein, is a high-yield bamboo plantation that can be grown sustainably.

Bamboo

Bamboo is a plant in the grass family Poaceae. Individual bamboo shoots emerge from the ground and grow to their full height in a single growing season. During the growing season, each new shoot grows vertically into a culm. Branching out does not occur until the majority of the mature height is reached, then branches can extend from nodes and leafing out occurs. Over time, the pulpy wall of each culm slowly hardens into a fully mature culm.

Some bamboo species exhibit a monopodial growth pattern. Monopodial bamboo species are also called running bamboos. Monopodial bamboo species have a underground root and rhizome system that spreads laterally. Individual plants merge into each other with no above ground distinction of where a single plant ends and the next plant begins. Individual culms emerge from the ground at random.

Some bamboo species exhibit a sympodial growth pattern. Sympodial bamboo species are also called clumping bamboos. Sympodial bamboo species have a underground root and rhizome system maintained within a relatively compact area. New culms emerge from this central system without significant lateral spread, and each bamboo plant or clump grows in an area that can be distinguished from a neighbouring plant or clump.

In one embodiment, sympodial bamboo species belong to the genus Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

In one embodiment, native or naturalized species of bamboo can be used in the plantation. The availability of a native species of high commercial value is a factor taken into consideration for a bamboo plantation. Where no suitable native species occurs, bamboo species can be selected by their ability to meet the restoration goals of the proposed project and area.

As with trees, each species of bamboo is unique, and has properties suitable for specific end uses. Yield and productivity, sugar content, silica content, wall thickness, fiber length, fiber density can vary between bamboo species.

Bamboo Plantations

In certain aspects, the invention relates to a bamboo plantation where the bamboo is integrated with pre-existing non-bamboo vegetation. The integration of pre-existing non-bamboo vegetation and bamboo results in a high-yielding bamboo plantation. This can allow for the commercial production of bamboo. Maintaining ecosystem integrity is critical for the long term productivity of bamboo. The bamboo plantation described herein recreates a natural ecosystem, maximizing productivity and maintaining ecosystem health.

Traditionally, bamboo plantations are planted as purely bamboo ecosystems. However, even if more than one bamboo species is planted, pure bamboo plantations reduce the health of the ecosystem and threaten the long term viability and yield of the plantations. Furthermore, bamboo monocultures exhibit lower yields. The bamboo plantation described herein maintains the long term health of the ecosystem, ensuring maximum and long term yield and can result in at least 20% higher yields than planting pure bamboo plantations.

Bamboo seed is highly rare, therefore, availability of propagational material has historically been one of the limiting factors for the development of commercial plantations. When propagational material is available, bamboo plantations developed from it have the ability to produce sustainable annual yields throughout the length of the plants genetic life time, which represents a time span of 60-120 years. The bamboo plantation describe herein can also be grown from a variety of propagational materials, such as tissue culture plantlets, vegetative propagation, as well as seed.

In certain aspects, the invention relates to a planting mechanism for bamboo species in which sympodial bamboo plants are planted in a uniform manner between pre-existing non-bamboo vegetation (e.g. trees, forest patches, native vegetation). The surrounding patches of non-uniform pre-existing non-bamboo vegetation can act as wildlife and biodiversity corridors. The invention results in a high-yielding plantation that shows diversity between the bamboo, and the non-bamboo vegetation species (e.g. trees, forest patches, native vegetation), which are maintained for conservation and biodiversity purposes and to enable the entire plantation to represent a close to nature planted ecosystem. The non-bamboo vegetation (e.g., trees, forest patches, native vegetation) is of different heights, composition and structure to the bamboo plantation area. This creates a complex three-dimensional environment that maximizes biodiversity and reduces the susceptibility of the ecosystem to pests or disease and increasing yields.

In certain aspects, the invention relates to a bamboo plantation comprising pre-existing non-bamboo vegetation and rows of sympodial bamboo plants, wherein the pre-existing vegetation comprises at least 10% of the total area of the plantation, and wherein the sympodial bamboo plants are planted at least 1 meter apart, and wherein the rows of sympodial bamboo plants are spaced at least 2 meters apart.

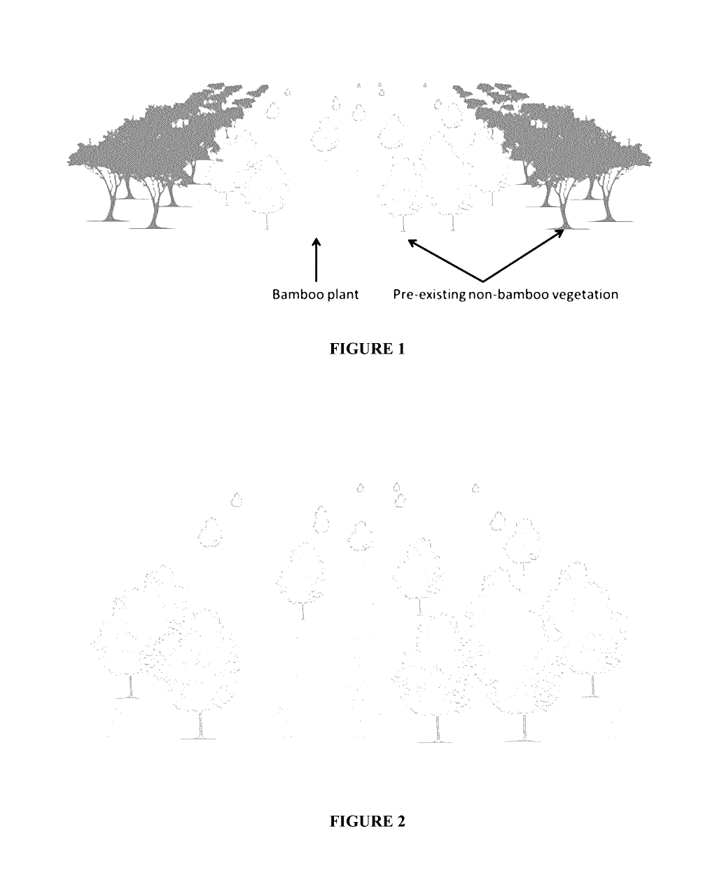

In some embodiments, the pre-existing non-bamboo vegetation, includes, but is not limited to standing trees, patches of forest, native vegetation, or remnant native ecosystems. For example, FIGS. 1 and 2 show embodiments of a bamboo plantation where the bamboo is integrated with pre-existing non-bamboo vegetation, such as forest (FIG. 1) or standing trees (FIG. 2). Rows of sympodial bamboo plants are integrated with the pre-existing non-bamboo vegetation.

In some embodiments, the sympodial bamboo plants are planted in a uniform manner between pre-existing non-bamboo vegetation (e.g., trees, forest patches, native vegetation). In some embodiments, the rows of sympodial bamboo plants are planted in regular spacing, with sympodial bamboo plants planted at least 1 meter apart. In some embodiments, the pre-existing non-bamboo vegetation is not removed to plant the sympodial bamboo plant. If pre-existing non-bamboo vegetation is present within the planting spot, the vegetation is left standing and the next regular spot is planted with the sympodial bamboo plant.

In some embodiments, the sympodial bamboo plants are spaced in rows, with the sympodial bamboo plants are planted at least 1 meter apart. In some embodiments, the sympodial bamboo plants are planted from 1 to 10 meters apart. In some embodiments, the sympodial bamboo plants are planted at least 2 meters apart. In some embodiments, the sympodial bamboo plants are planted at least 3 meters apart. In some embodiments, the sympodial bamboo plants are planted at least 4 meters apart. In some embodiments, the sympodial bamboo plants are planted at least 5 meters apart. In some embodiments, the sympodial bamboo plants are planted at least 6 meters apart. In some embodiments, the sympodial bamboo plants are planted at least 7 meters apart. In some embodiments, the sympodial bamboo plants are planted at least 8 meters apart. In some embodiments, the sympodial bamboo plants are planted at least 9 meters apart. In some embodiments, the sympodial bamboo plants are planted at least 10 meters apart. In some embodiments, all the sympodial bamboo plants in a row are planted the same distance apart from each other. In some embodiments, all the sympodial bamboo plants in a contiguous area are planted the same distance apart from each other.

In some embodiments, the sympodial bamboo plants are planted about 1 meter apart. In some embodiments, the sympodial bamboo plants are planted about 2 meters apart. In some embodiments, the sympodial bamboo plants are planted about 3 meters apart. In some embodiments, the sympodial bamboo plants are planted about 4 meters apart. In some embodiments, the sympodial bamboo plants are planted about 5 meters apart. In some embodiments, the sympodial bamboo plants are planted about 6 meters apart. In some embodiments, the sympodial bamboo plants are planted about 7 meters apart. In some embodiments, the sympodial bamboo plants are planted about 8 meters apart. In some embodiments, the sympodial bamboo plants are planted about 9 meters apart. In some embodiments, the sympodial bamboo plants are planted about 10 meters apart.

In some embodiments, the rows of sympodial bamboo plants are spaced at least 2 meters apart. In some embodiments, the rows of sympodial bamboo plants are spaced from 2 to 8 meters apart. In some embodiments, the rows of sympodial bamboo plants are spaced at least 3 meters apart. In some embodiments, the rows of sympodial bamboo plants are spaced at least 4 meters apart. In some embodiments, the rows of sympodial bamboo plants are spaced at least 5 meters apart. In some embodiments, the rows of sympodial bamboo plants are spaced at least 6 meters apart. In some embodiments, the rows of sympodial bamboo plants are spaced at least 7 meters apart. In some embodiments, the rows of sympodial bamboo plants are spaced at least 8 meters apart. In some embodiments, all the rows of sympodial bamboo plants in the plantation are planted the same distance apart from each other. In some embodiments, all the rows of sympodial bamboo plants in a contiguous area are planted the same distance apart from each other.

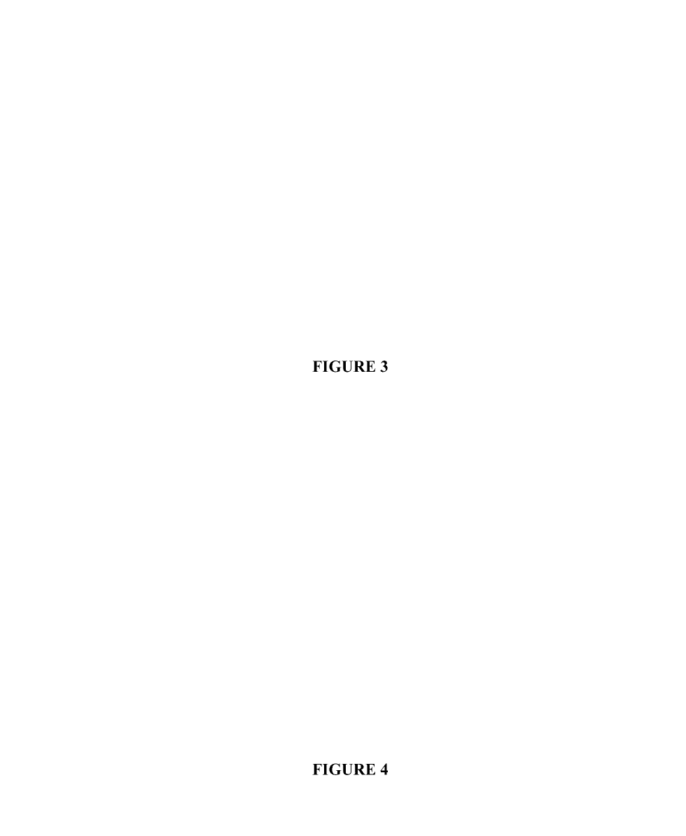

In some embodiments, the rows of sympodial bamboo plants are spaced about 2 meters apart. In some embodiments, the rows of sympodial bamboo plants are spaced about 3 meters apart. In some embodiments, the rows of sympodial bamboo plants are spaced about 4 meters apart. In some embodiments, the rows of sympodial bamboo plants are spaced about 5 meters apart. In some embodiments, the rows of sympodial bamboo plants are spaced about 6 meters apart. In some embodiments, the rows of sympodial bamboo plants are spaced about 7 meters apart. In some embodiments, the rows of sympodial bamboo plants are spaced about 8 meters apart. For example, FIGS. 3 and 4 show embodiments of a bamboo plantation comprising pre-existing non-bamboo vegetation and rows of sympodial bamboo plants. FIG. 3 shows sympodial bamboo plants planted at least 1 meter apart, and wherein the rows of sympodial bamboo plants are spaced at least 2 meters apart. FIG. 4 shows sympodial bamboo plants planted at least 10 meters apart, and wherein the rows of sympodial bamboo plants are spaced at least 8 meters apart.

In some embodiments, the rows of sympodial bamboo plants are planted parallel to each other. In some embodiments, individual sympodial bamboo plants within two adjacent rows do not need to be aligned.

In some embodiments, the pre-existing vegetation comprises at least 10% of the total area of the plantation. In some embodiments, the pre-existing non-bamboo vegetation comprises at least 20% of the total area of the plantation. In some embodiments, the pre-existing non-bamboo vegetation comprises at least 30% of the total area of the plantation. In some embodiments, the pre-existing non-bamboo vegetation comprises at least 40% of the total area of the plantation. In some embodiments, the pre-existing non-bamboo vegetation comprises at least 50% of the total area of the plantation.

In some embodiments, the pre-existing vegetation comprises about 10% of the total area of the plantation. In some embodiments, the pre-existing non-bamboo vegetation comprises about 20% of the total area of the plantation. In some embodiments, the pre-existing non-bamboo vegetation comprises about 30% of the total area of the plantation. In some embodiments, the pre-existing non-bamboo vegetation comprises about 40% of the total area of the plantation. In some embodiments, the pre-existing non-bamboo vegetation comprises about 50% of the total area of the plantation.

In some embodiments, the density of pre-existing non-bamboo vegetation is not uniform within the plantation, therefore, the plantation can comprise areas of sympodial bamboo plants integrated with pre-existing non-bamboo vegetation surrounded with areas of only pre-existing non-bamboo vegetation. In some embodiments, at least one area of the plantation comprises only pre-existing non-bamboo vegetation. In these areas of the plantation, the pre-existing non-bamboo vegetation is too dense to plant sympodial bamboo plants at least 1 meter apart without removing the existing vegetation. In other areas of the plantation, there may be no or sparse pre-existing non-bamboo vegetation and the area can be planted with regularly spaced sympodial bamboo plants. In some embodiments, the sympodial bamboo plants are planted at an average density over the total area of the plantation of at least 75 plants per acre. In some embodiments, the sympodial bamboo plants are planted at an average density over the total area of the plantation of about 75 plants per acre.

In some embodiments, at least one area of the plantation comprises only pre-existing non-bamboo vegetation. In some embodiments, at least two areas of the plantation comprises only pre-existing non-bamboo vegetation. In some embodiments, at least three areas of the plantation comprises only pre-existing non-bamboo vegetation. In some embodiments, at least four areas of the plantation comprises only pre-existing non-bamboo vegetation. In some embodiments, at least five areas of the plantation comprises only pre-existing non-bamboo vegetation. In some embodiments, at least six areas of the plantation comprises only pre-existing non-bamboo vegetation. In some embodiments, at least seven areas of the plantation comprises only pre-existing non-bamboo vegetation. In some embodiments, at least eight areas of the plantation comprises only pre-existing non-bamboo vegetation. In some embodiments, at least nine areas of the plantation comprises only pre-existing non-bamboo vegetation. In some embodiments, at least ten areas of the plantation comprises only pre-existing non-bamboo vegetation. In some embodiments, more than ten areas of the plantation comprises only pre-existing non-bamboo vegetation.

In some embodiments, one area of the plantation comprises only pre-existing non-bamboo vegetation. In some embodiments, two areas of the plantation comprises only pre-existing non-bamboo vegetation. In some embodiments, three areas of the plantation comprises only pre-existing non-bamboo vegetation. In some embodiments, four areas of the plantation comprises only pre-existing non-bamboo vegetation. In some embodiments, five areas of the plantation comprises only pre-existing non-bamboo vegetation. In some embodiments, six areas of the plantation comprises only pre-existing non-bamboo vegetation. In some embodiments, seven areas of the plantation comprises only pre-existing non-bamboo vegetation. In some embodiments, eight areas of the plantation comprises only pre-existing non-bamboo vegetation. In some embodiments, nine areas of the plantation comprises only pre-existing non-bamboo vegetation. In some embodiments, ten areas of the plantation comprises only pre-existing non-bamboo vegetation.

In some embodiments, the plantation comprises pre-existing non-bamboo vegetation and one species of sympodial bamboo plants. In some embodiments, the total area of the plantation is at least 1 acre. In some embodiments, one species of sympodial bamboo plants is used contiguously, separated only by the pre-existing non-bamboo vegetation, for at least 1 acre.

In some embodiments, the plantation comprises pre-existing non-bamboo vegetation and more than one species of sympodial bamboo plants. In some embodiments, the sympodial bamboo plants comprise more than one species of sympodial bamboo, wherein one species of sympodial bamboo is planted in an area of the plantation and each area is at least 1 acre. In some embodiments, the sympodial bamboo plants comprise more than one species of sympodial bamboo, wherein each different species of sympodial bamboo is planted in a different area of the plantation and each area is at least 1 acre. In some embodiments, the sympodial bamboo plants comprise more than two species of sympodial bamboo. In some embodiments, the different sympodial bamboo plant species are not intercropped. For example, in some embodiments, a first species of sympodial bamboo is planted in a first area of the plantation, and a second species of sympodial bamboo is planted in a second area of the plantation. In some embodiments, the first area and the second area are at least 1 acre. In some embodiments, each different species of sympodial bamboo plants is used contiguously, separated only by the pre-existing non-bamboo vegetation, for at least 1 acre.

In some embodiments, the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

Planting of rows of sympodial bamboo plants, integrated with pre-existing non-bamboo vegetation has been found to increase the productivity of the plantation by at least 20%. In some embodiments, the plantation yields at least 60 metric tons of raw green bamboo biomass per hectare per year. In some embodiments, the plantation yields at least 70 metric tons of raw green bamboo biomass per hectare per year. In some embodiments, the plantation yields at least 80 metric tons of raw green bamboo biomass per hectare per year. In some embodiments, the plantation yields at least 90 metric tons of raw green bamboo biomass per hectare per year.

In some embodiments, the plantation yields about 60 metric tons of raw green bamboo biomass per hectare per year. In some embodiments, the plantation yields about 70 metric tons of raw green bamboo biomass per hectare per year. In some embodiments, the plantation yields about 80 metric tons of raw green bamboo biomass per hectare per year. In some embodiments, the plantation yields about 90 metric tons of raw green bamboo biomass per hectare per year.

In some embodiments, the plantation yields at least 20% more raw green bamboo biomass per hectare per year than a bamboo plantation comprising only bamboo plants. In some embodiments, the plantation yields at least 20% more raw green bamboo biomass per hectare per year than a bamboo plantation comprising less than 10% non-bamboo vegetation. In some embodiments, the plantation yields at least 30% more raw green bamboo biomass per hectare per year. In some embodiments, the plantation yields at least 40% more raw green bamboo biomass per hectare per year. In some embodiments, the plantation yields at least 50% more raw green bamboo biomass per hectare per year. In some embodiments, the plantation yields at least 60% more raw green bamboo biomass per hectare per year. In some embodiments, the plantation yields about 20% to about 60% more raw green bamboo biomass per hectare per year. In some embodiments, the plantation yields about 20% to about 30% more raw green bamboo biomass per hectare per year. In some embodiments, the plantation yields about 30% to about 40% more raw green bamboo biomass per hectare per year. In some embodiments, the plantation yields about 40% to about 50% more raw green bamboo biomass per hectare per year. In some embodiments, the plantation yields about 50% to about 60% more raw green bamboo biomass per hectare per year.

In certain aspects, the invention relates to a bamboo plantation grown from tissue culture plantlets, vegetative propagation, or from seed. In some embodiments, the sympodial bamboo plants are planted from seed, tissue culture plantlets or by vegetative means.

In some embodiments, the plantation is farmed using conventional methods. In some embodiments, the sympodial bamboo plants are farmed using conventional methods, including but not limited to fertilization, weeding and pest control. In some embodiments, the pre-existing non-bamboo vegetation is not managed or farmed in any way (e.g. no fertilization, weeding and pest control is performed). In some embodiments, the plantations have reduced susceptibility to pests or disease.

Methods of Planting and Growing a Bamboo Plantation

In certain aspects, the invention relates to a method of planting a bamboo plantation, comprising planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area of the plantation.

In certain aspects, the invention relates to a method of growing sympodial bamboo, comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In some embodiments, the pre-existing non-bamboo vegetation, includes, but is not limited to standing trees, patches of forest, native vegetation, or remnant native ecosystems.

In some embodiments, the sympodial bamboo plants are planted in a uniform manner between pre-existing non-bamboo vegetation (e.g., trees, forest patches, native vegetation). In some embodiments, the rows of sympodial bamboo plants are planted in regular spacing, with sympodial bamboo plants planted at least 1 meter apart. In some embodiments, the pre-existing non-bamboo vegetation is not removed to plant the sympodial bamboo plant. If pre-existing non-bamboo vegetation is present within the planting spot, the vegetation is left standing and the next regular spot is planted with the sympodial bamboo plant.

In some embodiments, the sympodial bamboo plants are spaced in rows, with the sympodial bamboo plants are planted at least 1 meter apart. In some embodiments, the sympodial bamboo plants are planted from 1 to 10 meters apart. In some embodiments, the sympodial bamboo plants are planted at least 2 meters apart. In some embodiments, the sympodial bamboo plants are planted at least 3 meters apart. In some embodiments, the sympodial bamboo plants are planted at least 4 meters apart. In some embodiments, the sympodial bamboo plants are planted at least 5 meters apart. In some embodiments, the sympodial bamboo plants are planted at least 6 meters apart. In some embodiments, the sympodial bamboo plants are planted at least 7 meters apart. In some embodiments, the sympodial bamboo plants are planted at least 8 meters apart. In some embodiments, the sympodial bamboo plants are planted at least 9 meters apart. In some embodiments, the sympodial bamboo plants are planted at least 10 meters apart. In some embodiments, all the sympodial bamboo plants in a row are planted the same distance apart from each other. In some embodiments, all the sympodial bamboo plants in a contiguous area are planted the same distance apart from each other.

In some embodiments, the sympodial bamboo plants are planted about 1 meter apart. In some embodiments, the sympodial bamboo plants are planted about 2 meters apart. In some embodiments, the sympodial bamboo plants are planted about 3 meters apart. In some embodiments, the sympodial bamboo plants are planted about 4 meters apart. In some embodiments, the sympodial bamboo plants are planted about 5 meters apart. In some embodiments, the sympodial bamboo plants are planted about 6 meters apart. In some embodiments, the sympodial bamboo plants are planted about 7 meters apart. In some embodiments, the sympodial bamboo plants are planted about 8 meters apart. In some embodiments, the sympodial bamboo plants are planted about 9 meters apart. In some embodiments, the sympodial bamboo plants are planted about 10 meters apart.

In some embodiments, the rows of sympodial bamboo plants are spaced at least 2 meters apart. In some embodiments, the rows of sympodial bamboo plants are spaced from 2 to 8 meters apart. In some embodiments, the rows of sympodial bamboo plants are spaced at least 3 meters apart. In some embodiments, the rows of sympodial bamboo plants are spaced at least 4 meters apart. In some embodiments, the rows of sympodial bamboo plants are spaced at least 5 meters apart. In some embodiments, the rows of sympodial bamboo plants are spaced at least 6 meters apart. In some embodiments, the rows of sympodial bamboo plants are spaced at least 7 meters apart. In some embodiments, the rows of sympodial bamboo plants are spaced at least 8 meters apart. In some embodiments, all the rows of sympodial bamboo plants in the plantation are planted the same distance apart from each other. In some embodiments, all the rows of sympodial bamboo plants in a contiguous area are planted the same distance apart from each other.

In some embodiments, the rows of sympodial bamboo plants are spaced about 2 meters apart. In some embodiments, the rows of sympodial bamboo plants are spaced about 3 meters apart. In some embodiments, the rows of sympodial bamboo plants are spaced about 4 meters apart. In some embodiments, the rows of sympodial bamboo plants are spaced about 5 meters apart. In some embodiments, the rows of sympodial bamboo plants are spaced about 6 meters apart. In some embodiments, the rows of sympodial bamboo plants are spaced about 7 meters apart. In some embodiments, the rows of sympodial bamboo plants are spaced about 8 meters apart.

In some embodiments, the rows of sympodial bamboo plants are planted parallel to each other. In some embodiments, individual sympodial bamboo plants within two adjacent rows do not need to be aligned.

In some embodiments, the pre-existing vegetation comprises at least 10% of the total area of the plantation. In some embodiments, the pre-existing non-bamboo vegetation comprises at least 20% of the total area of the plantation. In some embodiments, the pre-existing non-bamboo vegetation comprises at least 30% of the total area of the plantation. In some embodiments, the pre-existing non-bamboo vegetation comprises at least 40% of the total area of the plantation. In some embodiments, the pre-existing non-bamboo vegetation comprises at least 50% of the total area of the plantation.

In some embodiments, the pre-existing vegetation comprises about 10% of the total area of the plantation. In some embodiments, the pre-existing non-bamboo vegetation comprises about 20% of the total area of the plantation. In some embodiments, the pre-existing non-bamboo vegetation comprises about 30% of the total area of the plantation. In some embodiments, the pre-existing non-bamboo vegetation comprises about 40% of the total area of the plantation.

In some embodiments, the density of pre-existing non-bamboo vegetation is not uniform within the plantation, therefore, the plantation can comprise areas of sympodial bamboo plants integrated with pre-existing non-bamboo vegetation surrounded with areas of only pre-existing non-bamboo vegetation. In some embodiments, at least one area of the plantation comprises only pre-existing non-bamboo vegetation. In these areas of the plantation, the pre-existing non-bamboo vegetation is too dense to plant sympodial bamboo plants at least 1 meter apart without removing the existing vegetation. In other areas of the plantation, there may be no or sparse pre-existing non-bamboo vegetation and the area can be planted with regularly spaced sympodial bamboo plants. In some embodiments, the sympodial bamboo plants are planted at an average density over the total area of the plantation of at least 75 plants per acre. In some embodiments, the sympodial bamboo plants are planted at an average density over the total area of the plantation of about 75 plants per acre.

In some embodiments, at least one area of the plantation comprises only pre-existing non-bamboo vegetation. In some embodiments, at least two areas of the plantation comprises only pre-existing non-bamboo vegetation. In some embodiments, at least three areas of the plantation comprises only pre-existing non-bamboo vegetation. In some embodiments, at least four areas of the plantation comprises only pre-existing non-bamboo vegetation. In some embodiments, at least five areas of the plantation comprises only pre-existing non-bamboo vegetation. In some embodiments, at least six areas of the plantation comprises only pre-existing non-bamboo vegetation. In some embodiments, at least seven areas of the plantation comprises only pre-existing non-bamboo vegetation. In some embodiments, at least eight areas of the plantation comprises only pre-existing non-bamboo vegetation. In some embodiments, at least nine areas of the plantation comprises only pre-existing non-bamboo vegetation. In some embodiments, at least ten areas of the plantation comprises only pre-existing non-bamboo vegetation. In some embodiments, more than ten areas of the plantation comprises only pre-existing non-bamboo vegetation.

In some embodiments, one area of the plantation comprises only pre-existing non-bamboo vegetation. In some embodiments, two areas of the plantation comprises only pre-existing non-bamboo vegetation. In some embodiments, three areas of the plantation comprises only pre-existing non-bamboo vegetation. In some embodiments, four areas of the plantation comprises only pre-existing non-bamboo vegetation. In some embodiments, five areas of the plantation comprises only pre-existing non-bamboo vegetation. In some embodiments, six areas of the plantation comprises only pre-existing non-bamboo vegetation. In some embodiments, seven areas of the plantation comprises only pre-existing non-bamboo vegetation. In some embodiments, eight areas of the plantation comprises only pre-existing non-bamboo vegetation. In some embodiments, nine areas of the plantation comprises only pre-existing non-bamboo vegetation. In some embodiments, ten areas of the plantation comprises only pre-existing non-bamboo vegetation.

In some embodiments, the plantation comprises pre-existing non-bamboo vegetation and one species of sympodial bamboo plants. In some embodiments, the total area of the plantation is at least 1 acre. In some embodiments, one species of sympodial bamboo plants is used contiguously, separated only by the pre-existing non-bamboo vegetation, for at least 1 acre.

In some embodiments, the plantation comprises pre-existing non-bamboo vegetation and more than one species of sympodial bamboo plants. In some embodiments, the sympodial bamboo plants comprise more than one species of sympodial bamboo, wherein one species of sympodial bamboo is planted in an area of the plantation and each area is at least 1 acre. In some embodiments, the sympodial bamboo plants comprise more than one species of sympodial bamboo, wherein each different species of sympodial bamboo is planted in a different area of the plantation and each area is at least 1 acre. In some embodiments, the sympodial bamboo plants comprise more than two species of sympodial bamboo. In some embodiments, the different sympodial bamboo plant species are not intercropped. For example, in some embodiments, a first species of sympodial bamboo is planted in a first area of the plantation, and a second species of sympodial bamboo is planted in a second area of the plantation. In some embodiments, the first area and the second area are at least 1 acre. In some embodiments, each different species of sympodial bamboo plants is used contiguously, separated only by the pre-existing non-bamboo vegetation, for at least 1 acre.

In some embodiments, the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp. In some embodiments, the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

Planting of rows of sympodial bamboo plants, integrated with pre-existing non-bamboo vegetation has been found to increase the productivity of the plantation by at least 20%. In some embodiments, the plantation yields at least 60 metric tons of raw green bamboo biomass per hectare per year. In some embodiments, the plantation yields at least 70 metric tons of raw green bamboo biomass per hectare per year. In some embodiments, the plantation yields at least 80 metric tons of raw green bamboo biomass per hectare per year. In some embodiments, the plantation yields at least 90 metric tons of raw green bamboo biomass per hectare per year.

In some embodiments, the plantation yields about 60 metric tons of raw green bamboo biomass per hectare per year. In some embodiments, the plantation yields about 70 metric tons of raw green bamboo biomass per hectare per year. In some embodiments, the plantation yields about 80 metric tons of raw green bamboo biomass per hectare per year. In some embodiments, the plantation yields about 90 metric tons of raw green bamboo biomass per hectare per year.

In some embodiments, the plantation yields at least 20% more raw green bamboo biomass per hectare per year than a bamboo plantation comprising only bamboo plants. In some embodiments, the plantation yields at least 20% more raw green bamboo biomass per hectare per year than a bamboo plantation comprising less than 10% non-bamboo vegetation. In some embodiments, the plantation yields at least 30% more raw green bamboo biomass per hectare per year. In some embodiments, the plantation yields at least 40% more raw green bamboo biomass per hectare per year. In some embodiments, the plantation yields at least 50% more raw green bamboo biomass per hectare per year. In some embodiments, the plantation yields at least 60% more raw green bamboo biomass per hectare per year. In some embodiments, the plantation yields about 20% to about 60% more raw green bamboo biomass per hectare per year. In some embodiments, the plantation yields about 20% to about 30% more raw green bamboo biomass per hectare per year. In some embodiments, the plantation yields about 30% to about 40% more raw green bamboo biomass per hectare per year. In some embodiments, the plantation yields about 40% to about 50% more raw green bamboo biomass per hectare per year. In some embodiments, the plantation yields about 50% to about 60% more raw green bamboo biomass per hectare per year.

In certain aspects, the invention relates to a method of planting a bamboo plantation using tissue culture plantlets, vegetative propagation, or from seed. In some embodiments, the sympodial bamboo plants are planted from seed, tissue culture plantlets or by vegetative means.

In some embodiments, the plantation is farmed using conventional methods. In some embodiments, the sympodial bamboo plants are farmed using conventional methods, including but not limited to fertilization, weeding and pest control. In some embodiments, the pre-existing non-bamboo vegetation is not managed or farmed in any way (e.g. no fertilization, weeding and pest control is performed). In some embodiments, the plantations have reduced susceptibility to pests or disease.

In some embodiments, the sympodial bamboo is planted on degraded land. Land degradation can be caused by multiple forces, including extreme weather conditions, and human activities that over use, pollute or degrade the quality of soils and the resulting productive capacity of the land. Land degradation can negatively affecting food production, livelihoods, and the production and provision of other ecosystem goods and services. Land degradation has accelerated during the 20th century due to increasing and combined pressures of agricultural and livestock production (over-cultivation, overgrazing, forest conversion), urbanization, deforestation, and extreme weather events. Land restoration is the process of restoring land back to its natural state. In some embodiments, growing sympodial bamboo on the degraded land restores the ecosystem functions of the degraded land.

Uses of Bamboo

In some embodiments, bamboo can be used as an alternative fiber for pulp, paper (e.g., toilet paper, toweling, facial tissues, kitchen tissues, cleaning tissues, napkins), textiles, carbonized products (e.g., activated carbon, charcoal, shisha charcoal), and engineered timber. In some embodiments, the sympodial bamboo plants of the bamboo plantation can be harvested to provide the raw green biomass to produce bamboo based products. In some embodiment, the sympodial bamboo plants are harvested annually.

In certain aspects, the invention relates to bamboo pulp prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to paper prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to textiles prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to activated carbon prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants.

In certain aspects, the invention relates to engineered timber prepared from bamboo grown by the method comprising (a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and (b) cultivating the sympodial bamboo plants. In some embodiments, the engineered timber is a composite timber, a plywood or a veneer.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention, and are covered by the following claims.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

FIG. 5 shows a 3 year old plantation in Nicaragua where the sympodial bamboo Guadua aculeata plants have been planted 6.25 meters apart, and wherein the rows of sympodial bamboo plants are spaced at 6.25 meters apart. In FIG. 5, conserved forest and native vegetation is shown as an example of pre-existing non-bamboo vegetation

### Example 2

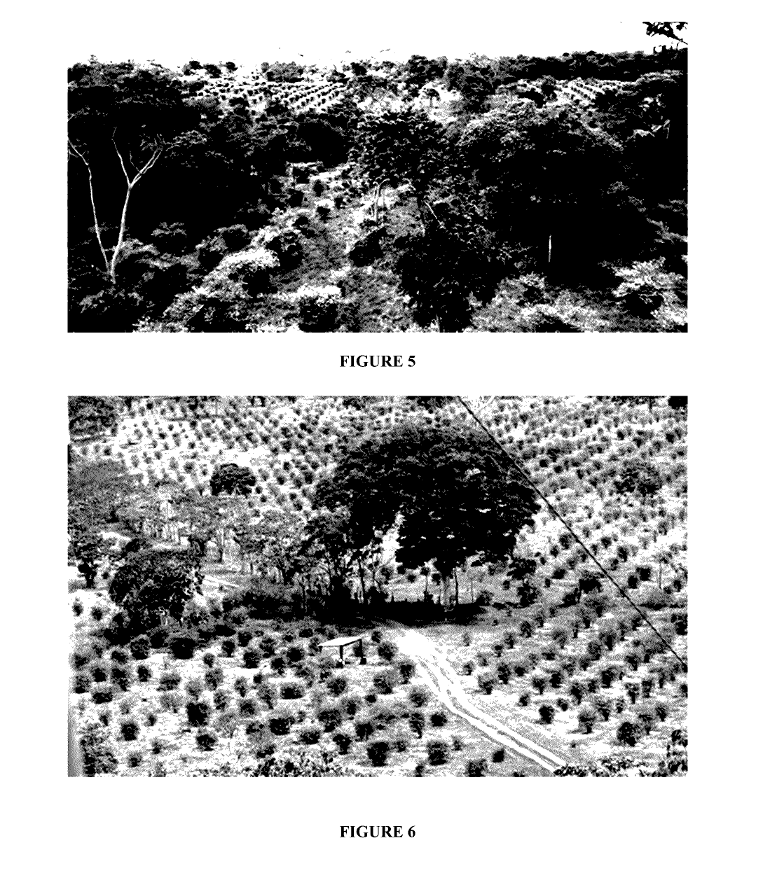

FIG. 6 shows a 2 year old plantation in Nicaragua where the sympodial bamboo Guadua aculeata plants have been planted 5 meters apart, and wherein the rows of sympodial bamboo plants are spaced at 5 meters apart. In FIG. 6, standing trees are shown as an example of pre-existing non-bamboo vegetation.

### Example 3

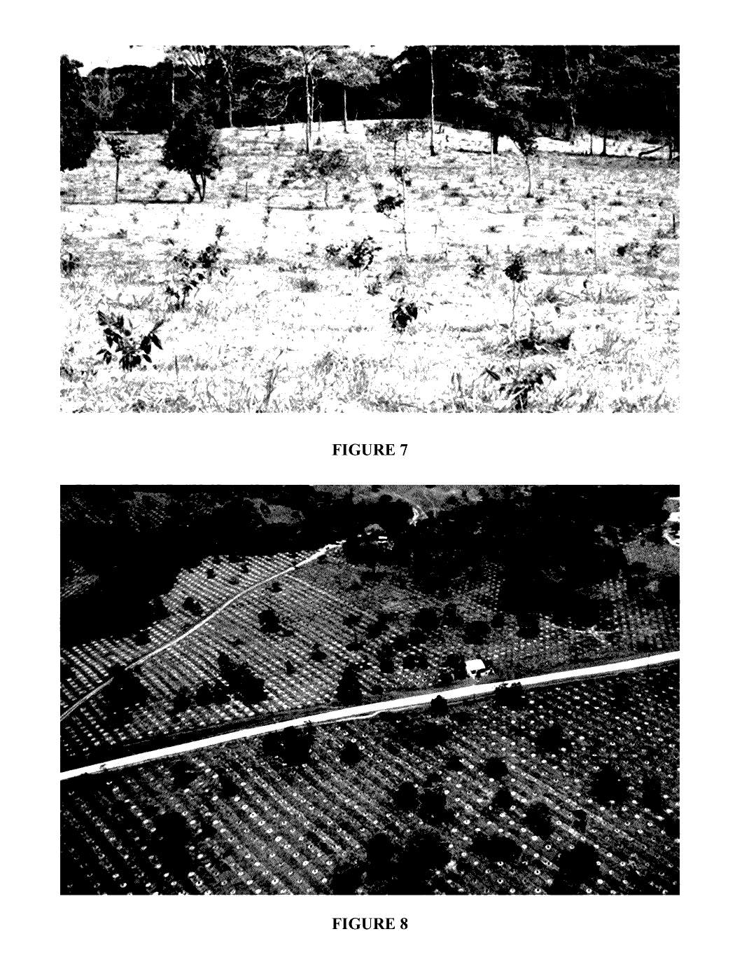

FIG. 7 shows a newly planted plantation where the sympodial bamboo Guadua aculeata plants have been planted at least 5 meters apart, and wherein the rows of sympodial bamboo plants are spaced at least 5 meters apart. In FIG. 7, both conserved forest and standing trees shown as an example of pre-existing non-bamboo vegetation.

### Example 4

FIG. 8 shows an aerial view of a plantation where sympodial bamboo plants have been planted at a density of at least 175 plants per acre. In FIG. 8, conserved forest and standing trees are shown as an example of pre-existing non-bamboo vegetation.

### Example 5

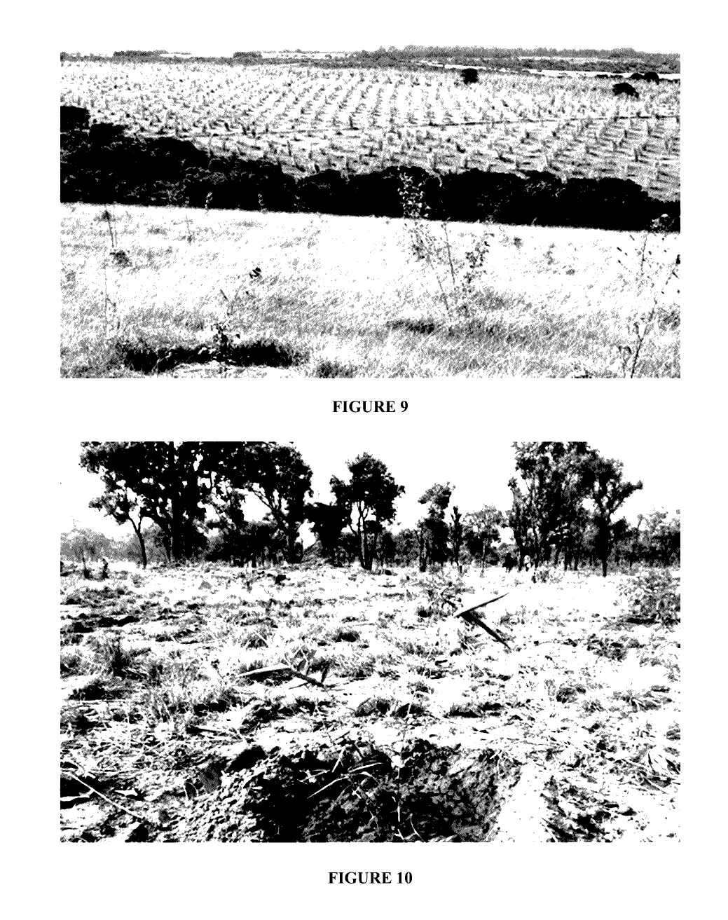

FIG. 9 shows a 1 year old plantation in South Africa where the sympodial bamboo Bambusa balcooa plants have been planted 5 meters apart, and wherein the rows of sympodial bamboo plants are spaced at 5 meters apart. In FIG. 9, conserved native vegetation is shown as an example of pre-existing non-bamboo vegetation.

### Example 6

FIG. 10 shows a newly planted plantation in Ghana where the sympodial bamboo Dendrocalamus asper plants have been planted 5 meters apart, and wherein the rows of sympodial bamboo plants are spaced at least 3 meters meters apart. In FIG. 10, conserved forest and standing trees are shown as an example of pre-existing non-bamboo vegetation.

1. A bamboo plantation comprising pre-existing non-bamboo vegetation and rows of sympodial bamboo plants, wherein the pre-existing vegetation comprises at least 10% of the total area of the plantation, and wherein the sympodial bamboo plants are planted at least 1 meter apart, and wherein the rows of sympodial bamboo plants are spaced at least 2 meters apart.

2. The bamboo plantation of claim 1, wherein the sympodial bamboo plants are planted at an average density over the total area of the plantation of at least 75 plants per acre.

3. The bamboo plantation of claim 1, wherein the sympodial bamboo plants comprise one species of sympodial bamboo.

4. The bamboo plantation of claim 1, wherein the total area of the plantation is at least 1 acre.

5. The bamboo plantation of claim 1, wherein the sympodial bamboo plants comprise more than one species of sympodial bamboo, wherein one species of sympodial bamboo is planted in an area of the plantation and each area is at least 1 acre.

6. The bamboo plantation of claim 1, wherein the pre-existing non-bamboo vegetation comprises at least 20% of the total area of the plantation.

7. The bamboo plantation of claim 1, wherein the pre-existing non-bamboo vegetation comprises at least 30% of the total area of the plantation.

8. The bamboo plantation of claim 1, wherein the pre-existing non-bamboo vegetation comprises at least 40% of the total area of the plantation.

9. The bamboo plantation of claim 1, wherein the pre-existing non-bamboo vegetation comprises at least 50% of the total area of the plantation.

10. The bamboo plantation of claim 1, wherein the density of pre-existing non-bamboo vegetation is not uniform within the plantation.

11. The bamboo plantation of claim 1, wherein the pre-existing non-bamboo vegetation is not removed to plant the sympodial bamboo plant.

12. The bamboo plantation of claim 1, wherein at least one area of the plantation comprises only pre-existing non-bamboo vegetation.

13. The bamboo plantation of claim 1, wherein the sympodial bamboo plants are planted from 1 to 10 meters apart.

14. The bamboo plantation of claim 1, wherein the rows of sympodial bamboo plants are spaced from 2 to 8 meters apart.

15. The bamboo plantation of claim 1, wherein the plantation yields at least 60 metric tons of raw green bamboo biomass per hectare per year.

16. The bamboo plantation of claim 1, wherein the plantation yields at least 20% more raw green bamboo biomass per hectare per year than a bamboo plantation comprising only bamboo plants.

17. The bamboo plantation of claim 1, wherein the plantation yields at least 20% more raw green bamboo biomass per hectare per year than a bamboo plantation comprising less than 10% non-bamboo vegetation.

18. The bamboo plantation of claim 16 or 17, wherein the plantation yields at least 30% more raw green bamboo biomass per hectare per year.

19. The bamboo plantation of claim 16 or 17, wherein the plantation yields at least 40% more raw green bamboo biomass per hectare per year.

20. The bamboo plantation of claim 16 or 17, wherein the plantation yields at least 50% more raw green bamboo biomass per hectare per year.

21. The bamboo plantation of claim 16 or 17, wherein the plantation yields at least 60% more raw green bamboo biomass per hectare per year.

22. The bamboo plantation of claim 16 or 17, wherein the plantation yields 20% to 60% more raw green bamboo biomass per hectare per year.

23. The bamboo plantation of claim 16 or 17, wherein the plantation yields 20% to 30% more raw green bamboo biomass per hectare per year.

24. The bamboo plantation of claim 16 or 17, wherein the plantation yields 30% to 40% more raw green bamboo biomass per hectare per year.

25. The bamboo plantation of claim 16 or 17, wherein the plantation yields 40% to 50% more raw green bamboo biomass per hectare per year.

26. The bamboo plantation of claim 16 or 17, wherein the plantation yields 50% to 60% more raw green bamboo biomass per hectare per year.

27. The bamboo plantation of claim 1, wherein the sympodial bamboo belongs to the genus: Bambusa sp., Cephalostachym sp., Chusquea sp., Dendrocalamus sp., Dinochloa sp., Fargesia sp., Gigantochloa sp., Guadua sp., Melocalamus sp., Oxytenanthera sp., Schizostachyum sp., Thamnocalamus sp., Thyrostachys sp., or Yushania sp.

28. The bamboo plantation of claim 27, wherein the sympodial bamboo is Bambusa vulgaris, Bambusa balcooa, Bambusa blumeana, Bambusa polymorpha, Bambusa textilis, Cephalostachyum pergracile, Dendrocalamus asper, Dendrocalamus barbatus, Dendrocalamus giganteus, Dendrocalamus membrenaceus cv grandis, Dendrocalamus strictus, Dendrocalamus sinicus, Guadua aculeata, Guadua angustifolia, or Oxytenanthera abyssinica.

29. A method of planting a bamboo plantation, comprising planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area of the plantation.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A method of growing sympodial bamboo, comprising:
a) planting rows of sympodial bamboo plants between pre-existing non-bamboo vegetation, wherein the sympodial bamboo plants are planted at least 1 meter apart, and wherein the rows of sympodial bamboo plants are spaced at least 2 meters apart, and wherein the pre-existing non-bamboo vegetation comprises at least 10% of the total area being planted; and
b) cultivating the sympodial bamboo plants.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. Bamboo pulp prepared from bamboo grown by the method of claim 43.

72. Paper prepared from bamboo grown by the method of claim 43.

73. Textiles prepared from bamboo grown by the method of claim 43.

74. Activated carbon prepared from bamboo grown by the method of claim 43.

75. Engineered timber prepared from bamboo grown by the method of claim 43.

76. (canceled)
